(19) United States
(12) Patent Application Publication
(10) Pub. No.: US 2026/0272891 A1
(43) Pub. Date: Sep. 17, 2026

(54) TREATMENT OF AUTOIMMUNE DISEASES AND CANCER WITH PROTEASE INHIBITORS

(71) Applicant: Lundquist Institute for Biomedical Innovation at Harbor-UCLA Medical Center, Torrance, CA (US)

(72) Inventor: Delphine J. Lee, Torrance, CA (US)

(21) Appl. No.: 19/469,224

(22) PCT Filed: Apr. 5, 2024
(86) PCT No.: PCT/US2024/023321

§ 371 Date: Sep. 25, 2025

Related U.S. Application Data
(60) Provisional application No. 63/494,647, filed on Apr. 6, 2023.

(51) Int. Cl. A61K 31/427; A61K 31/403; A61P 17/06; A61P 35/00

(52) U.S. Cl. CPC A61K 31/427; A61K 31/403; A61P 17/06; A61P 35/00

(57) ABSTRACT

The present disclosure provides methods and uses for reducing immunity, suppressing or modulating immune reaction. Such modulation of immunity and immune reaction can be useful for preventing or treating diseases such as cancers, inflammatory disorders, and autoimmune or autoinflammatory diseases or conditions. The methods and uses can entail administer to a subject in need thereof one or more protease inhibitors, optionally along with a CYP3A4 inhibitor.

### CROSS-REFERENCE TO RELATED APPLICATIONS

The present application claims the priority benefit of U.S. Provisional Application No. 63/494,647, filed Apr. 6, 2023, which is hereby incorporated by reference in its entirety.

### BACKGROUND

In December 2021, US Food and Drug Administration (FDA) authorized for emergency use of Paxlovid, a combination of two drugs: nirmatrelvir (NMV), a SARS-COV-2 main protease inhibitor associated with ritonavir (RTV), an HIV-1 protease inhibitor and CYP3A inhibitor, for treatment of mild-to-moderate COVID-19 in adults and pediatric patients (12 years old and at least 40 kg weight). Paxlovid significantly reduced the proportion of people with COVID-19 related hospitalization or death from any cause by 88% compared to placebo. Currently, Paxlovid is still considered an investigational drug, although is available at many pharmacies. Therefore, more research on future development and the applications of this drug and potential other effects and applications of its use are warranted.

Antiviral chemotherapeutic interventions often target specific viral enzymes or attack a weak point of viral replication within the host, such as targeting the divergent RNA-dependent RNA polymerase. They are developed based on the viral life cycle, which comprehends four mechanisms: 1: binding to receptors (mABS, convalescent plasma or plasma-derived therapies and vaccines fall within this category), 2: entry-exit, as preventing viral/host interaction and endosome maturation, such as ACE2 inhibitors and human protease inhibitors); 3: proteolysis, as preventing of protein formation involved in viral replication with protease inhibitors and 4: RNA replication, as preventing of viral genome replication through RNA polymerase inhibitors. Protease inhibitors are precedent to treat hepatitis C (HCV) and human immunodeficiency viruses (HIV), by blocking the essential processing of viral proteins for replication. Studies suggest that protease inhibitors work clinically at concentrations above those shown to eradicate 90% of the virus in in vitro assays.

As mentioned, NMV is a protease inhibitor that blocks the virus from replicating, while Ritonavir contributes to keep NMV at higher concentrations in the body for a longer time period by inhibiting its metabolism. NMV was designed to inhibit Main protease (Mpro). Mpro is the viral papain-like protease (PLpro) and the 3C-like cysteine protease (3 CLpro). Mpro is required to produce the non-structural proteins (nsp) 1-16 of SARS-COV-2, one of the most attractive drug targets among coronaviruses, since its essential for viral replication and its substrate-recognition pocket is highly conserved. NMV binds to the active site of Mpro and forms a reversible covalent bond with the catalytic cysteine residue, Cys145. Inhibitors of Mpro render the protein incapable of processing polyprotein precursors, thereby preventing viral replication. NMV showed to have antiviral activity against all variants of concern, including Omicron and its variants. (Yang et al., (2005). PLOS Biol. Oct;3(10):e 324; Owen et al., (2021). Science. Dec 24;374(6575):1586-1593; Razali et al., (2021) Microorganisms. Nov 30;9(12):2481; Yang et al., (2022). J Med Chem. Jul 14;65(13):8686-8698; Ahmad et al., (2021). Int J Mol Sci. Aug 24;22(17):9124; Vangeel et al., (2022). Antiviral Res. Feb:198:105252). Ritonavir is an oral antiretroviral medication originally developed to treat HIV that constitutes a strong CYP3A inhibitor (Cooper et al., (2003). Clin Infect Dis. Jun 15;36(12):1585-92.). It lacks activity against SARS-COV-2 Mpro but enhances the bioavailability of NMV by slowing its degradation by CYP34A (Owen et al., (2021). Science. Dec 24;374(6575):1586-1593; Blair, (2023). Drugs Ther Perspect. 39(2):41-47). Most common side effects are observed at high doses (600 mg BID); hence, only a low dose is used in combination with other protease inhibitors as a pharmacokinetic (PK) enhancer (Cooper et al., (2003). Clin Infect Dis. Jun 15;36(12):1585-92).

### SUMMARY

While protease inhibitors are in clinical use, neither of these drugs (NMV nor RTV) have been described to modulate inflammatory responses to treat conditions mediated by inflammatory cytokines in inflammatory/autoimmune skin disorders. Interestingly, an unexpected effect of NMV+RTV was discovered on activated peripheral blood mononuclear cells, indicating its ability to modulate inflammatory cytokine secretion (see Example 1) and potential efficacy in skin inflammations (Example 2). NMV alone or NMV+RTV also kills cancer cells in a dose dependent matter (Example 3). Thus, NMV and/or RTV, or their combination, may be employed for treatment of immune diseases, such as skin inflammation, cancers and other diseases.

The present disclosure provides methods for reducing immunity, suppressing or modulating immune reaction, or preventing or treating cancer, an inflammatory disorder, or an autoimmune or autoinflammatory disease or condition in a subject in need thereof, comprising administering to the subject a protease inhibitor.

In certain embodiments, the one or more protease inhibitors provided herein regulate inflammation to treat autoimmune and autoinflammatory skin diseases since immunoproteasome activity has shown to be involved in the progression of various autoimmune diseases.

In certain embodiments, the protease inhibitor is a viral protease inhibitor.

In certain embodiments, the protease inhibitor is a 3-chymotrypsin-like protease inhibitor. In certain embodiments, the protease inhibitor is nirmatrelvir, ensitrelvir, boceprevir, paritaprevir, ombitasvir, tipranavir, ivermectin, simnotrelvir, and micafungin. In certain embodiments, the protease inhibitor is nirmatrelvir.

In certain embodiments, the protease inhibitor further comprises CYP3A4 inhibitor. In certain embodiments, the CYP3A4 inhibitor comprises amiodarone, amlodipine, amprenavir, aprepitant, atazanavir, azamulin, berberine, berberis, bergamottin, bicalutamide, boceprevir, buprenorphine, cafestol, cannabidiol, carambola, ceritinib, chloramphenicol, chlorzoxazone, ciclosporin, cilostazol, cimetidine, ciprofloxacin, clarithromycin, clotrimazole, cobicistat, conivaptan, crizotinib, delavirdine, 6′,7′-dihydroxybergamottin, diltiazem, dithiocarbamate, dronedarone, econazole, erythromycin, fluconazole, fluvoxamine, fosamprenavir, fosaprepitant, gestodene, idelalisib, imatinib, indinavir, isavuconazonium, isoniazid, istradefylline, itraconazole, ivacaftor, ketoconazole, levoketoconazole, lomitapide, lopinavir, methoxsalen, mibefradil, miconazole, mifepristone, nefazodone, nelfinavir, niacin, nicotinamide, norfloxacin, omeprazole, orphenadrine, piperine, posaconazole, quercetin, ranitidine, ranolazine, ribociclib, ritonavir, rutin, saquinavir, sesamin, silybum marianum, tacrolimus, telaprevir, telithromycin, ticagrelor, tofisopam, troleandomycin, tucatinib, valerian, valproate, verapamil, vitamin B3, and voriconazole. In certain embodiments, the protease inhibitor is ritonavir.

In certain embodiments, the protease inhibitor comprises nirmatrelvir and ritonavir.

In certain embodiments, the subject is an organ transplantation patient.

In certain embodiments, the subject has received or is prescribed for an immune cell therapy, preferably selected from the group consisting an allogeneic or autologous T cell, NK cell, or macrophage therapy.

In certain embodiments, the cell comprises a chimeric antigen receptor or a recombinant T cell receptor.

In certain embodiments, the subject has cytokine release syndrome.

In certain embodiments, the subject has an autoimmune or autoinflammatory disease.

In certain embodiments, the autoimmune disease is selected from the group consisting of rheumatoid arthritis, systemic lupus erythematosus (SLE), multiple sclerosis (MS), atherosclerosis, amyotrophic lateral sclerosis, type 1 diabetes, psoriasis, atopic dermatitis, contact dermatitis and eczematous dermatitis, osteoarthritis, allergic airway disease (e.g. asthma), chronic obstructive pulmonary disease (COPD), inflammatory bowel disease (IBD), Crohn's disease, ulcerative colitis, cutaneous lupus erythrematosis, lupus nephritis, dermatomyositis, dry eye disease, Sjögren's syndrome, Myasthenia gravis, Grave's disease, Hashimoto's thyroiditis, Celiac disease, Pernicious anemia, autoimmune hemolytic anemia, Goodpasture syndrome, dermatomyositis, polymyositis, and Addison's disease.

In certain embodiments, the subject has acute or chronic inflammation.

In certain embodiments, the acute or chronic inflammation is at any tissue, preferably in the skin. In certain embodiments, the acute or chronic inflammation is infectious diseases, such as bacterial infection, viral infection or fungal infection, eczema (e.g., atopic, dyshidrotic, or seborrheic dermatitis), psoriasis (e.g., plaque, pustular, or inverse psoriasis), rosacea (e.g., phymatous or papulopustular rosacea), dermatomyositis, impetigo (non-bullous and bullous type), and vitiligo (generalized and non-segmental type), hand dermatitis, lichen planus, drug hypersensitivity reactions, lichen simplex chronicus, prurigo nodularis, cutaneous T cell lymphoma, blistering autoimmune diseases (pemphigus family of disorders or bullous pemphigoid/pemphigoid family of disorders), or retinoid dermatitis.

In certain embodiments, the treatment method provided herein further comprises topical administration of an agent selected from the group consisting of a moisturizer, a corticosteroid, an immunomodulator, dapsone, inhibitors of the JAK-STAT pathway, inhibitors of the IL-17 pathway, inhibitors of the TNFα pathway, inhibitors of the cytokine-cytokine receptor interaction, cyclooxygenase inhibitors, macrolide antibiotics such as tacrolimus or pimecrolimus, inhibitors of PDE such as crisoborale or apremilast, or retinoid such as tretinoin, retinol, retinaldehyde, tazarotene, adapalene and combinations thereof.

In certain embodiments, the treatment method provided herein comprises administration of 1200 mg of nirmatrelvir and 200 mg of ritonavir daily.

In certain embodiments, the treatment method provided herein comprises administration of less than 1000 mg of nirmatrelvir and less than 180 mg of ritonavir daily.

In certain embodiments, the subject has cancer.

In certain embodiments, the cancer is a solid tumor. In certain embodiments, the cancer is leukemia or lymphoma.

In certain embodiments, the cancer is selected from the group consisting of bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, esophageal cancer, ovarian cancer, renal cancer, melanoma, skin cancer, prostate cancer and thyroid cancer.

### BRIEF DESCRIPTION OF THE DRAWINGS

FIG. 1A shows FSC versus SSC histogram gating shows similarity in percentage of PBMCs among different donors with three different concentrations of NMV. PBMCs from Donor 186 treated with 1250 ng of NMV had a 52.8% of PBMCs while when treated with 5000 ng the counting was 47.7% and when treated with 2500 ng the cell showed 44.8%. negative control with DMSO only the counting was 44.6%. Donor 317, counting cells showed similar results when treated with 1250 ng of NMV the cell counting was 55.4%, with a increasing in cell counting when treated with 2500 ng, showing 64.4%. 5000 ng of NMV showed 55.9%. Donor 93, with 5000 ng, 2500 ng and 1250 ng respectively showed 54.6%, 76.2% and 74.8% when compared to DMSO only group with 54.9% cell percentage.

FIG. 1B shows CD3+ T cells from three different donors (D186, D317 and D93) were treated with NMV in three different concentrations: 1250 ng, 2500 ng and 5000 ng to verify drug toxicity. Toxicity of T cells were similar between the three groups. Donor 186: Negative control had 28.1% of dead cells while when cells were treated with 5000 ng of NMV showed 28.5% of cells double positive for Propidium iodide (PI) and CD3. When cells were treated with 2500 ng and 1250 ngm of NMV respectively, 31.1% and 29.9% of T cells showed disruption in membrane for staining with PI (DNA/RNA in dead cells). Donor 317: Negative control had 17.2% of dead cells while when cells were treated with 5000 ng of NMV showed 17.1% of cells double positive for Propidium iodide (PI) and CD3. When cells were treated with 2500 ng and 1250 ngm of NMV respectively, 16.9% and 17.9% of T cells showed disruption in membrane for staining with PI (DNA/RNA in dead cells). Donor 93: Negative control had 18.1% of dead cells while when cells were treated with 5000 ng of NMV showed 16.0% of cells double positive for Propidium iodide (PI) and CD3. When cells were treated with 2500 ng and 1250 ng of NMV respectively, 17.1% and 19.2% of T cells showed disruption in membrane for staining with PI (DNA/RNA in dead cells).

FIG. 1C shows CD14+ cells for monocytes from three different donors (D186, D317 and D93) were treated with NMV and three different concentrations: 1250 ng, 2500 ng and 5000 ng. to verify drug toxicity. Negative control was with DMSO only. Donor 186: Negative control had 0.49% of dead cells while when cells were treated with 5000 ng of NMV showed 0.74% of cells double positive for Propidium iodide (PI) and CD14. When cells were treated with 2500 ng and 1250 ng of NMV respectively, 1.28% and 0.75% of monocytes showed disruption in membrane for staining with PI (DNA/RNA in dead cells). Donor 317: Negative control had 0.34% of dead cells while when cells were treated with 5000 ng of NMV showed 0.32% of cells double positive for Propidium iodide (PI) and CD14. When cells were treated with 2500 ng and 1250 ngm of NMV respectively, 0.31% and 0.37% of monocytes cells showed disruption in membrane for staining with PI (DNA/RNA in dead cells). Donor 93: Negative control had 0.71% of dead cells while when cells were treated with 5000 ng of NMV showed 0.64% of cells double positive for Propidium iodide (PI) and CD14. When cells were treated with 2500 ng and 1250 ng of NMV respectively, 0.74% and 0.90% of CD14+ cells showed disruption in membrane for staining positive with PI (DNA/RNA in dead cells).

FIG. 2A shows FSC versus SSC histogram gating shows similiarity in percentage of PBMCs among different donors with three different concentrations of RTV. PBMCs from Donor 186 treated with 1250 ng of RTV had a 48.0% of PBMCs while when treated with 5000 ng the counting was 44.9% and when treated with 2500 ng the cell showed 51.3%. negative control with DMSO only the counting was 50.3%. Donor 317, counting cells showed similar results when treated with 1250 ng of RTV the cell counting was 61.3%, with a decreasing in cell counting when treated with 2500 ng, showing 56.2%. 5000 ng of RTV showed 57.4%. Donor 93, with 5000 ng, 2500 ng and 1250 ng respectivelly showed 55.9%, 62.9% and 60.4% when compared to DMSO only group with 66.6% cell percentage.

FIG. 2B shows CD3+ T cells from three different donors (D186, D317 and D93) were treated with RTV in three different concentrations: 1250 ng, 2500 ng and 5000 ng to verify drug toxicity. Toxicity of CD3+T cells were similar between the three groups. Donor 186: Negative control had 28.5% of positive for dead cells while when cells were treated with 5000 ng of RTV showed 28.6% of cells double positive for Propidium iodide (PI) and CD3. When cells were treated with 2500 ng and 1250 ngm of RTV respectively, 28.9% and 27.9% of T cells showed disruption in membrane for staining with PI (DNA/RNA in dead cells). Donor 317: Negative control had 16.5% of dead cells while when cells were treated with 5000 ng of RTV showed 17.8% of cells double positive for Propidium iodide (PI) and CD3. When cells were treated with 2500 ng and 1250 ngm of RTV respectively, 17.5% and 18.1% of T cells showed disruption in membrane for staining with PI (DNA/RNA in dead cells). Donor 93: Negative control had 18.8% positive for dead cells while when cells were treated with 5000 ng of RTV showed 19.5% of cells double positive for Propidium iodide (PI) and CD3. When cells were treated with 2500 ng and 1250 ngm of RTV respectively, 19.8% and 19.5% of T cells showed disruption in membrane for staining with PI (DNA/RNA in dead cells).

FIG. 2C shows CD14+ cells for monocytes from three different donors (D186, D317 and D93) were treated with RTV and three different concentrations: 1250 ng, 2500 ng and 5000 ng. to verify drug toxicity. Negative control was with DMSO only. Donor 186: Negative control had 1.49% of dead cells while when cells were treated with 5000 ng of RTV showed 1.71% of cells double positive for Propidium iodide (PI) and CD14. When cells were treated with 2500 ng and 1250 ngm of RTV respectively, 1.71% and 2.14% of monocytes showed disruption in membrane for staining with PI (DNA/RNA in dead cells). Donor 317: Negative control had 0.41% of dead cells while when cells were treated with 5000 ng of RTV showed 0.44% of cells double positive for Propidium iodide (PI) and CD14. When cells were treated with 2500 ng and 1250 ngm of RTV respectively, 0.38% and 0.42% of monocytes cells showed disruption in membrane for staining with PI (DNA/RNA in dead cells). Donor 93: Negative control had 0.75% of dead cells while when cells were treated with 5000 ng of RTV showed 0.81% of cells double positive for Propidium iodide (PI) and CD14. When cells were treated with 2500 ng and 1250 ngm of RTV respectively, 1.12% and 0.90% of CD14+ cells showed disruption in membrane for staining positive with PI (DNA/RNA in dead cells).

FIG. 3 shows ELISA Cytokines when PBMC are treated with NMV+RTV stimulated by LPS. ELISA performed in frozen PBMCs from three donors D186, D317 and D93. Treatment with NMV+RTV and Nirmatrelvir (NMV) and Ritonavir (RTV) were added in three different concentrations to evaluate the dose response when stimulated with LPS. We evaluated the concentration of five cytokines: IL-6, IL-10, IL-23, IL-1β, and TNF-α. As expected, LPS alone (red) stimulated all five cytokines compared to media control (black), and production of LPS-stimulated IL-6, IL-10, IL-23 and TNF-α was diminished by the presence of NMV (green) in a dose responsive manner (P=<0.001, <0.001,0.003, and 0.019 respectively), while IL-1β increased (P=0.010). In contrast, the addition of RTV (blue symbols) to the LPS stimulation had no effect on all five cytokines (P=0.371, 0.378, 0.170, 0.898, and 0.608 for IL-6, IL-10, IL-23, IL-1β, and TNF-α respectively) and levels were similar to LPS stimulated cells in the absence of any drug at any dose of RTV. However, the combination of RTV+NMV (green symbols) showed a greater reduction of cytokine production compared to just NMV alone at 5000 ng/ml for IL-10 (P=0.008). The combination showed reduction of TNF-α in all three donors, and reduction of IL-6 in two of three donors; however, the variation between donors was greater than the reduction by the drug. Two of three donors already had no detectable IL-23 levels with NMV alone; all three had 0's after the combination. As for IL-1β, (which had dose-response increased with NMV alone), the combination drug showed lower concentration compared to NMV at 5000 ng/mL (P=0.032).

FIG. 4 shows ELISA Cytokines when Monocytes are treated with NMV+RTV stimulated by LPS. Evaluation of pro and anti-inflammatory cytokines using monocytes from 3 different donors(D186, D317, D93). NMV and RTV concentrations used were 5000 ng/mL. The graphic show a decrease in IL-6, TNF-α, IL-10, IL-23 when treated with NMV, RTV only and NMV+RTV. IL-16 levels corroborate with the data showing increasing levels when treated with NMV, RTV and NMV+RTV in donors 317 and 93. Slightly decreased was shown with donor186 when treated with RTV.

FIG. 5A shows activated T cells treated with NMV, RTV and NMV+RTV when stimulated with α-CD3/CD28 Dynabeads Human T-Activator. Cell Viability (%): Negative control: 1, Cells treated with NMV only: 0.797468354. Cells treated with RTV only:1.050632911, cells treated with NMV+RTV (N+R):0.84810127. Activated cells only (α-CD3/CD28):1.658227848, cells treated Nirmatrelvir+α-CD3/CD28:1.367088608, cells treated with Ritonavir+α-CD3/CD28:1.759493671, cells treated with NMV and RTV (N+R) and activated α-CD3/CD28:1.443037975.

FIG. 5B shows frozen leukopak PBMCs treated with NMV, RTV only, NMV+RTV (N+R), NMV with LPS stimulation, RTV with LPS stimulation, N+R with LPS stimulation. Cell Viability (%): negative control (DMSO): 1, NMV only:0.661354582, RTV only:0.920318725, cells treated with N+R: 1.007968127, Cells stimulated only with LPS:0.994023904, cells treated with NMV and stimulated with LPS: 0.880478088, cells treated with RTV only and stimulated with LPS:1.844621514, cells treated with NMV+RTV (N+R) and stimulated with LPS: 1.099601594.

FIG. 6 shows ELISA Cytokines when PBMCs treated are with LPS and NMV+RTV. Elisa assay was performed using 5 cytokines in fresh blood-PBMCs from three independent donors (D5, D7, D8). NMV and RTV concentrations used were 5000 ng/ml. IL-6 showed decreased levels in all donors, donor 5 showed IL-6 higher levels when stimulated only with LPS when compared to levels when NMV and RTV (N+R+LPS) were added. IL-10 also showed significant increase in LPS groups with decreasing levels 5× lower when treated with NMV and RTV (N+R+LPS). IL-23 production was none when compared to groups stimulated by LPS only. Donor 5 and D8 had similar production of TNF-α when stimulated by LPS while D7 had more than 2000 pg/mL in production when stimulated with LPS. On the other hand, all donors showed lower levels when treated with NMV and RTV (N+R+LPS). Il-1β showed higher levels when PBMCs were treated with NMV and RTV (N+R+LPS).

FIG. 7 shows ELISA test using T cell activation to evaluate the cytokines IL-2, IL-4, IL-10, TNF-α and IFN-γ. T cells were activated using beads in a manner that partially mimics stimulation by antigen-presenting cells. NMV and RTV concentrations used were 5000 ng/ml. IL-2 production was elevated in all groups with activated cells and treated with NMV and RTV when compared to negative control. IL-4 levels were higher in activated cells only (Bead), activated cells treated with RTV (Bead+RTV). When cells were activated and treated with NMV (Bead+Nir) levels were lower when compared to all groups with activated cells, RTV treatment only and activated cells with both drugs (Bead+N&R). Activated cells treated with both NMV and RTV (Bead+N&R) production of IL-4 were higher when compared to Bead+Nir group but lower when compared to activated cells only (Bead) and activated cells with RTV only (Bead+RTV) groups. DMSO vs. Ritonavir w/ Beads−p-value is 0.0153. DMSO vs. Beads only−p-value is 0.0173. IFN-γ showed higher levels in Beads groups only and Bead+RTV group, while Bead+Nirmatrelvir (Beads+NIR) and Beads+NMV and RTV (Bead N&R) the production of IFN-y were lower when compared to activated cells only (Beads) and activated cells with RTV (Bead+RTV). DMSO vs. Ritonavir w/ Beads−p-value is 0.0157, DMSO vs. Beads−p-value is 0.0199. IL10 Production were higher in activated cells only and activated cells+RTV. Activated cells+NMR (Beads+NIR) and Activated cells with both drugs (Bead+N&R) were lower. DMSO(Media) group vs. Beads−p-value is 0.0070. TNF-α showed high levels in production in activated cells only (bead) and activated cells with RTV (bead+RTV). Production of TNF-α were lower in activated cells+NMR (Bead+NIR) and activated cells with both drugs (Bead+N&R) when compared to other groups except negative control (media only). DMSO vs. Ritonavir w/ Beads−p-value is 0.0130. DMSO (Media) group vs. Beads only group−p-value is 0.0154.

FIG. 8 shows frozen PBMC from donors D317, D186 and D93 were stimulated with 4 different types of bacteria. Sonicated bacteria (0.1 μg) and LPS (10 ng/ml) were used to stimulate the inflammatory cytokines and treated with 5000 ng/ml of NMV+RTV. The figure shows decrease in cytokines IL-6, IL-10, IL-23 and TNF-α treated with NMV+RTV when in presence of LPS and M. Radiotolerans, S. yanoikuyae, C. acnes and C. granulosum. IL-1β was increased when stimulated with LPS and sonicated bacteria.

FIG. 9A and FIG. 9B show gene expression analysis by RNA sequencing. FIG. 9A shows exploratory analysis of gene expression profile by t-SNE ordination. Shading was at the 70% confidence interval. FIG. 9B shows venn-diagrams for pair-wise comparisons in the LPS with NMV+RTV experiment (top) and with anti-CD3/CD28 with NMV+RTV experiment (bottom), with numbers indiciating total Ensembl geneIDS differentially expressed (P<0.05). Blue are positive fold-changes and Red are negative fold-changes. NMV+RTV concentrations are in ng/mL.

FIG. 10, with panel A-C show Ritonavir alone (A), Nirmatrelvir alone (B), or Ritonavir+Nirmatrelvir (C) killed 4T1 cells in a dose responsive manner.

FIG. 11, with panel A-D show dose response manner of the lung cancer cell lines to Pax (Ritonavir+Nirmatrelvir), respectively.

FIG. 12, with panel A-C show dose response manner of the melanoma cell lines to Pax (Ritonavir+Nirmatrelvir), respectively. FIG. 12D shows dose response manner of the non-cancer melanocyte (Ritonavir+Nirmatrelvir).

### DETAILED DESCRIPTION

### Definition

As used in the present specification, the following words, phrases and symbols are generally intended to have the meanings as set forth below, except to the extent that the context in which they are used indicates otherwise.

In certain embodiments, as used herein, the phrase “one or more” refers to one to five. In certain embodiments, as used herein, the phrase “one or more” refers to one to three.

Any compound or structure given herein, is also intended to represent unlabeled forms as well as isotopically labeled forms of the compounds. These forms of compounds may also be referred to as “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

In many cases, the compounds of this disclosure are capable of forming acid and/or base salts by virtue of the presence of amino, and/or carboxyl groups, or groups similar thereto.

Provided are also or a pharmaceutically acceptable salt, isotopically enriched analog, deuterated analog, stereoisomer, mixture of stereoisomers, and prodrugs of the compounds described herein. “Pharmaceutically acceptable” or “physiologically acceptable” refer to compounds, salts, compositions, dosage forms, and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids, and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic or organic acids. Salts derived from inorganic acids include, e.g., hydrochloric acid, hydrobromic acid, sulfuric acid, nitric acid, phosphoric acid, and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic or organic bases. Salts derived from inorganic bases include, by way of example only, sodium, potassium, lithium, aluminum, ammonium, calcium, and magnesium salts. Salts derived from organic bases include, but are not limited to, salts of primary, secondary, and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

“Prodrugs” means any compound which releases an active parent drug according to a structure described herein in vivo when such prodrug is administered to a mammalian subject. Prodrugs of a compound described herein are prepared by modifying functional groups present in the compound described herein in such a way that the modifications may be cleaved in vivo to release the parent compound. Prodrugs may be prepared by modifying functional groups present in the compounds in such a way that the modifications are cleaved, either in routine manipulation or in vivo, to the parent compounds. Prodrugs include compounds described herein wherein a hydroxy, amino, carboxyl, or sulfhydryl group in a compound described herein is bonded to any group that may be cleaved in vivo to regenerate the free hydroxy, amino, or sulfhydryl group, respectively. Examples of prodrugs include, but are not limited to esters (e.g., acetate, formate, and benzoate derivatives), amides, guanidines, carbamates (e.g., N,N-dimethylaminocarbonyl) of hydroxy functional groups in compounds described herein, and the like. Preparation, selection, and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

The terms “optional” or “optionally” means that the subsequently described event or circumstance may or may not occur, and that the description includes instances where said event or circumstance occurs and instances in which it does not. Also, the term “optionally substituted” refers to any one or more hydrogen atoms on the designated atom or group may or may not be replaced by a moiety other than hydrogen.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human. The term “patient” is generally synonymous with the term “subject”.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition. The therapeutically effective amount may vary depending on the subject, and disease or condition being treated, the weight and age of the subject, the severity of the disease or condition, and the manner of administering, which can readily be determined by one or ordinary skill in the art.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

### Protease Inhibitors

Proteases play a major role in the posttranslational processing of proteins, protein catabolismand various pathological processes, and therefore, they become a natural target for protease inhibitors (Pis). Proteases are a diverse group of enzymes that are involved in a variety of biological processes such as organ growth, aging, cancer development, inflammation, healing and embryogenesis (Barrett, A., et al. (2003). “The Handbook of Proteolytic Enzymes.” Academic Press(2^{nd} ed).). Proteases and naturally occurring small molecule protease inhibitors maintain the functional and structural integrity of physiological systems, including the skin. Protease dysfunction activity may be associated with abnormalities and inflammation developed in the skin.

An expressive number of proteases are able to modulate many physiological conditions in the human skin, they are classified into six broad groups according to their catalytic domain: serine proteases, cysteine proteases, aspartate proteases, threonine proteases, glutamic acid proteases, and metalloproteases (also referred to as metalloproteinases). Protease inhibitors can regulate proteolytic activity in the skin, thus contributing to the integrity and protective barrier function of the skin. Changes or imbalances of enzymes or inhibitor levels in the skin (and other target organs) may result in inflammation, manifesting in skin diseases with signs and symptoms of redness, scaling, swelling, itching, etc. Proteases may be expressed endogenously by resident skin cells, by infiltrating immune cells, or from microorganisms, allergens, and other external sources.

While protease inhibitors are in clinical use, neither of these drugs (NMV nor RTV) have been described to modulate inflammatory responses to treat conditions mediated by inflammatory cytokines in inflammatory/autoimmune skin disorders.

The instant inventors made an unexpected finding that the combination of nirmatrelvir (NMV), a protease inhibitor, and ritonavir (RTV) which is also a protease inhibitor but, also importantly, induces CYP1A2 and inhibits CYP3A4 and CYP2D6, was able to modulate inflammatory cytokine secretion (see Example 1) and has potential efficacy in reducing skin inflammations (Example 2). NMV alone or NMV+RTV also kills cancer cells in a dose dependent matter (Example 3). Thus, NMV and/or RTV, or their combination, may be employed for treatment of immune diseases, such as skin inflammation, cancers and other diseases.

In accordance with one embodiment of the present disclosure, therefore, provided is a method for reducing immunity, suppressing or modulating immune reaction, or preventing or treating cancer, an inflammatory disorder, or an autoimmune or autoinflammatory disease or condition in a subject in need thereof, comprising administering to the subject a protease inhibitor.

In certain embodiments, the protease inhibitor is a viral protease inhibitor. The protease inhibitors (Pis) provided herein are usually used to treat viral infections, such as HIV/AIDS, hepatitis C and Covid-19, by preventing the virus from replication.

In certain embodiments, the viral protease inhibitor is selected from the group consisting of sofosbuvir, saquinavir, ritonavir, nilfinabvir, lopinavir, indinavir, glecaprevir, gabexate, darunabir, danoprevir, camostat, atazanavir, asunaprebir, amprenavir, umifenovir, tenofovir, temsavir, telaprevir, ribavirin, remdesivir, raltegravir, pleconaril, pimodvir, pibrentasvir, peramivir, oseltamvir, etravirine, nirmatrelvir, elvitegravir, delavirdine, abacavir, ribostamycin, quinine, posaconazole, methyIPrednisone, maraviroc, itraconazole, hydrochloroquine, fumagillin, chloroquine phosphate, candicidin, atrovastatin, boceprevir, paritaprevir and tipranavir, ivermectin, micafungin, ombitasvir.

In certain embodiments, the protease inhibitor is a 3-chymotrypsin-like protease (3Clpro) inhibitor. The 3Clpro inhibitor can include nirmatrelvir, ensitrelvir, boceprevir, paritaprevir, ombitasvir, tipranavir, ivermectin, simnotrelvir, and micafungin. In certain embodiments, the protease inhibitor is nirmatrelvir.

In certain embodiments, the protease inhibitor is a CYP3A4 inhibitor. The CYP3A4 inhibitor can include amiodarone, amlodipine, amprenavir, aprepitant, atazanavir, azamulin, berberine, berberis, bergamottin, bicalutamide, boceprevir, buprenorphine, cafestol, cannabidiol, carambola, ceritinib, chloramphenicol, chlorzoxazone, ciclosporin, cilostazol, cimetidine, ciprofloxacin, clarithromycin, clotrimazole, cobicistat, conivaptan, crizotinib, delavirdine, 6′,7′-dihydroxybergamottin, diltiazem, dithiocarbamate, dronedarone, econazole, erythromycin, fluconazole, fluvoxamine, fosamprenavir, fosaprepitant, gestodene, idelalisib, imatinib, indinavir, isavuconazonium, isoniazid, istradefylline, itraconazole, ivacaftor, ketoconazole, levoketoconazole, lomitapide, lopinavir, methoxsalen, mibefradil, miconazole, mifepristone, nefazodone, nelfinavir, niacin, nicotinamide, norfloxacin, omeprazole, orphenadrine, piperine, posaconazole, quercetin, ranitidine, ranolazine, ribociclib, ritonavir, rutin, saquinavir, sesamin, silybum marianum, tacrolimus, telaprevir, telithromycin, ticagrelor, tofisopam, troleandomycin, tucatinib, valerian, valproate, verapamil, vitamin B3, and voriconazole. In certain embodiments, the protease inhibitor is ritonavir.

In certain embodiments, the protease inhibitor used in the present disclosure is a combination of one or more protease inhibitors. In certain embodiments, the protease inhibitor comprises nirmatrelvir and ritonavir.

Methods of administration of the protease inhibitor or composition comprising the protease inhibitor include but are not limited to intradermal, intramuscular, intraperitoneal, intravenous, subcutaneous, intranasal, epidural, and oral routes. The protease inhibitor or composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the protease inhibitor of the disclosure may be administered orally, rectally, parenterally, intracistemally, intravaginally, intraperitoneally, topically (as by powders, ointments, drops or transdermal patch), bucally, or as an oral or nasal spray.

The term “parenteral” as used herein refers to modes of administration which include intravenous, intramuscular, intraperitoneal, intrasternal, subcutaneous and intra-articular injection and infusion.

Administration can be systemic or local. In addition, it may be desirable to introduce the protease inhibitor of the disclosure into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It may be desirable to administer the compositions of the disclosure locally to the area in need of treatment; this may be achieved by, for example, and not by way of limitation, local infusion during surgery, topical application, e.g., in conjunction, with a wound dressing after surgery, by injection, by means of a catheter, by means of a suppository, or by means of an implant, said implant being of a porous, non-porous, or gelatinous material, including membranes, such as sialastic membranes, or fibers.

### Combinations and Compositions

The present disclosure also provides pharmaceutical compositions. Such compositions comprise an effective amount of one or more protease inhibitors, and an acceptable carrier. In some embodiments, the composition includes a protease inhibitor of the instant disclosure, and a CYP3A4 inhibitor.

In some embodiments, the protease inhibitor is selected from the group consisting of sofosbuvir, saquinavir, ritonavir, nilfinabvir, lopinavir, indinavir, glecaprevir, gabexate, darunabir, danoprevir, camostat, atazanavir, asunaprebir, amprenavir, umifenovir, tenofovir, temsavir, telaprevir, ribavirin, remdesivir, raltegravir, pleconaril, pimodvir, pibrentasvir, peramivir, oseltamvir, etravirine, nirmatrelvir, elvitegravir, delavirdine, abacavir, ribostamycin, quinine, posaconazole, methyIPrednisone, maraviroc, itraconazole, hydrochloroquine, fumagillin, chloroquine phosphate, candicidin, atrovastatin, boceprevir, paritaprevir and tipranavir, ivermectin, micafungin, ombitasvir.

The CYP3A4 inhibitor, in some embodiments, can include amiodarone, amlodipine, amprenavir, aprepitant, atazanavir, azamulin, berberine, berberis, bergamottin, bicalutamide, boceprevir, buprenorphine, cafestol, cannabidiol, carambola, ceritinib, chloramphenicol, chlorzoxazone, ciclosporin, cilostazol, cimetidine, ciprofloxacin, clarithromycin, clotrimazole, cobicistat, conivaptan, crizotinib, delavirdine, 6′,7′-dihydroxybergamottin, diltiazem, dithiocarbamate, dronedarone, econazole, erythromycin, fluconazole, fluvoxamine, fosamprenavir, fosaprepitant, gestodene, idelalisib, imatinib, indinavir, isavuconazonium, isoniazid, istradefylline, itraconazole, ivacaftor, ketoconazole, levoketoconazole, lomitapide, lopinavir, methoxsalen, mibefradil, miconazole, mifepristone, nefazodone, nelfinavir, niacin, nicotinamide, norfloxacin, omeprazole, orphenadrine, piperine, posaconazole, quercetin, ranitidine, ranolazine, ribociclib, ritonavir, rutin, saquinavir, sesamin, silybum marianum, tacrolimus, telaprevir, telithromycin, ticagrelor, tofisopam, troleandomycin, tucatinib, valerian, valproate, verapamil, vitamin B3, and voriconazole.

In some embodiments, the combination or composition includes nirmatrelvir and ritonavir.

In some embodiments, the composition further includes a second agent (e.g., a moisturizer, a corticosteroid, an immunomodulator, dapsone, inhibitors of the JAK-STAT pathway, inhibitors of the IL-17 pathway, inhibitors of the KEGG pathway, inhibitors of the cytokine-cytokine receptor interaction, cyclooxygenase inhibitors, macrolide antibiotics such as tacrolimus or pimecrolimus, inhibitors of PDE such as crisoborale or apremilast, or retinoid such as tretinoin, retinol, retinaldehyde, tazarotene, adapalene and combinations thereof).

Compounds provided herein are usually administered in the form of pharmaceutical compositions. Thus, provided herein are also pharmaceutical compositions that contain one or more of the compounds described herein or a pharmaceutically acceptable salt, tautomer, stereoisomer, mixture of stereoisomers, prodrug, or deuterated analog thereof and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17^{th} Ed. (1985); and Modern Pharmaceutics, Marcel Dekker, Inc. 3^{rd} Ed. (G.S. Banker & C.T. Rhodes, Eds.).

The pharmaceutical compositions may be administered in either single or multiple doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein or a pharmaceutically acceptable salt, tautomer, stereoisomer, mixture of stereoisomers, prodrug, or deuterated analog thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein or a pharmaceutically acceptable salt, tautomer, stereoisomer, mixture of stereoisomers, prodrug, or deuterated analog thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, tautomer, stereoisomer, mixture of stereoisomers, prodrug, or deuterated analog thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound such as the one or more protease inhibitors described herein administered per dose or per day. Daily dosage of a compound may be between about 1 mg and 5,000 mg, about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

When administered orally, the total daily dosage for a human subject may be between 1 mg and 1,000 mg, between about 1,000-2,000 mg/day, between about 10-500 mg/day, between about 50-300 mg/day, between about 75-200 mg/day, or between about 100-150 mg/day. In certain embodiments, the total daily dosage for a human subject may be 50 mg, 100 mg, 150 mg, 200 mg, 250 mg, 300 mg, 350 mg, 400 mg, 450 mg, 500 mg, 550 mg, 600 mg, 650 mg, 700 mg, 800 mg, 850 mg, 900 mg, 950 mg, 1000 mg, 1500 mg, or 2000 mg.

The compounds of the present application or the compositions thereof may be administered once, twice, three, or four times daily, using any suitable mode described above. Also, administration or treatment with the compounds may be continued for a number of days; for example, commonly treatment would continue for at least 3 days, 5 days, 7 days, 14 days, or 28 days, for one cycle of treatment.

In a particular embodiment, the method comprises administering to the subject an initial daily dose of about 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week, or once per week.

In certain embodiments, the total daily dosage for a human subject may be 50 mg, 100 mg, 150 mg, 200 mg, 250 mg, 300 mg, 350 mg, 400 mg, 450 mg, 500 mg, 550 mg, 600 mg, 650 mg, 700 mg, 800 mg, 850 mg, 900 mg, 950 mg, 1000 mg, 1500 mg, or 2000 mg. The compounds of the present application or the compositions thereof may be administered once, twice, three, or four times daily, using any suitable mode described above. The pharmaceutical compositions containing the protease inhibitor of the disclosure may be administered orally, rectally, parenterally, intracistemally, intravaginally, intraperitoneally, topically (as by powders, ointments, drops or transdermal patch), bucally, or as an oral or nasal spray.

### Immunity and Inflammation

In some embodiments, provided are methods for preventing or treating an inflammatory disorder, or an autoimmune or autoinflammatory disease or condition in a subject in need thereof. The method, in one embodiment, entails administering to the patient an effective amount of a protease inhibitor of the present disclosure.

In some embodiments, provided are uses of the protease inhibitors of the present disclosure in the manufacture of a medicament for preventing or treating an inflammatory disorder, or an autoimmune or autoinflammatory disease or condition in a subject in need thereof.

In some embodiments, provided are the protease inhibitor of the present disclosure for use in the prevention or treatment of an inflammatory disorder, or an autoimmune or autoinflammatory disease or condition in a subject in need thereof.

In some embodiments, provided are methods for reducing immunity, suppressing or modulating immune reaction in a subject in need thereof. The method, in one embodiment, entails administering to the patient an effective amount of a protease inhibitor of the present disclosure.

In some embodiments, provided are uses of the protease inhibitors of the present disclosure in the manufacture of a medicament for reducing immunity, suppressing or modulating immune reaction in a subject in need thereof.

In some embodiments, provided are the protease inhibitor of the present disclosure for use in the reduction of immunity, suppression or modulation of immune reaction in a subject in need thereof.

The innate and adaptive immune systems can be categorized into 3 major kinds of cell-mediated effector immunity, based on different effector T-cell and innate lymphoid cell (ILC) lineages: type 1, type 2, and type 3. Type 1 immunity consists of T-bet+IFN-γ-producing group 1 ILCs (ILC1 and natural killer cells), CD8+ cytotoxic T cells (TC1), and CD4+ TH1 cells, which protect against intracellular microbes through activation of mononuclear phagocytes. Type 2 immunity consists of GATA-3+ ILC2s, TC2 cells, and TH2 cells producing IL-4, IL-5, and IL-13, which induce mast cell, basophil, and eosinophil activation, as well as IgE antibody production, thus protecting against helminthes and venoms. Type 3 immunity is mediated by retinoic acid-related orphan receptor γt+ ILC3s, TC17 cells, and TH17 cells producing IL-17, IL-22, or both, which activate mononuclear phagocytes but also recruit neutrophils and induce epithelial antimicrobial responses, thus protecting against extracellular bacteria and fungi. On the other hand, type 1 and 3 immunity mediate autoimmune diseases, whereas type 2 responses can cause allergic diseases. The immunity or immune reaction of the present disclosure includes type 1, type 2, and/or type 3 immunity.

Inflammation or inflammatory disorder is a biological response of the immune system that can be triggered by a variety of factors, including pathogens, damaged cells, toxic compounds, and irradiation. These factors may induce acute and/or chronic inflammatory responses in the skin, heart, pancreas, liver, kidney, lung, brain, intestinal tract and reproductive system, potentially leading to tissue damage or disease. Both infectious and non-infectious agents and cell damage activate inflammatory cells and trigger inflammatory signaling pathways, most commonly the NF-κB, MAPK, and JAK-STAT pathways.

Usually, during acute inflammatory responses, cellular and molecular events and interactions efficiently minimize impending injury or infection. This mitigation process contributes to restoration of tissue homeostasis and resolution of the acute inflammation. However, uncontrolled acute inflammation may become chronic, contributing to a variety of chronic inflammatory diseases.

Various pathogenic factors, such as infection, tissue injury, or cardiac infarction, can induce inflammation by causing tissue damage. The etiologies of inflammation can be infectious or non-infectious. In response to tissue injury, the body initiates a chemical signaling cascade that stimulates responses aimed at healing affected tissues. These signals activate leukocyte chemotaxis from the general circulation to sites of damage. These activated leukocytes produce cytokines that induce inflammatory responses. Inflammation is a common pathogenesis of many chronic diseases, including cardiovascular and bowel diseases, diabetes, arthritis, and cancer.

In certain embodiments, the subject has an immunity disease or inflammatory disorder.

### Autoimmune and Autoinflammatory

Over a hundred years ago, the concept of autoimmune reaction against the organism's own elements was established by Paul Ehrlich. The classification of autoimmunity has become more complex since the rapid evolution of the understanding of genetics and the molecular mechanisms involved in the pathophysiology, which has challenged the old autoimmunity paradigm of classification. In addition, recent efforts to reappraise autoimmunity takes into account the genes and cells involved in certain types of autoimmunity and other diseases that are not strictly autoimmune but are autoinflammatory. Differences between autoimmunity and autoinflammation are as follows: autoimmunity is a self-directed inflammation caused by aberrant dendritic cells and T and B cell behaviors that disrupt tolerance, resulting in an adaptive immune response that plays a central role in the phenotypical clinical expression of autoimmune diseases. In sharp contrast, autoinflammation leads to the activation of the innate immunity and may result in tissue damage through the alteration of cytokine cascades, which induce site-specific inflammation and is independent of the adaptive immune response. In addition to understanding autoimmunity classification, it is important to understand tolerance mechanisms and the reasons why physiological control is disrupted.

In certain embodiments, the subject has an autoimmune or autoinflammatory disease.

Autoimmune diseases may include rheumatoid arthritis, systemic lupus erythematosus (SLE), multiple sclerosis (MS), atherosclerosis, amyotrophic lateral sclerosis, type 1 diabetes, psoriasis, atopic dermatitis, contact dermatitis and eczematous dermatitis, osteoarthritis, allergic airway disease (e.g. asthma), chronic obstructive pulmonary disease (COPD), inflammatory bowel disease (IBD), Crohn's disease, ulcerative colitis, cutaneous lupus erythrematosis, lupus nephritis, dermatomyositis, dry eye disease, Sjögren's syndrome, Myasthenia gravis, Grave's disease, Hashimoto's thyroiditis, Celiac disease, Pernicious anemia, autoimmune hemolytic anemia, Goodpasture syndrome, dermatomyositis, polymyositis, and Addison's disease.

Based on the observation of a patient reporting improvement of skin inflammation while taking Paxlovid, Paxlovid was contemplated to affect immune cell cytokine secretion in vitro. Others have previously shown that protease inhibitors (Pis) can reduce the pro-inflammatory cytokine environment (Galli et al., (2019). Autoimmun Rev. Oct;18(10):102370; Delmonte et al., (2007). Immunol Lett. Aug 15;111(2):111-5). A study of cells from mice showed the PI Ritonavir dampened the cytotoxic activity of T cells by modulating the immunoproteasome chymotrypsin-like activity and impairing the epitope presentation by MHC I molecules as well as decreasing IL2, IFN-gamma and TNF-alpha secretion (André et al., (1998). PNAS. Oct 27;95(22):13120-4.). The immunoproteasome is expressed in hematopoietic cells and synthesized in upon various stresses, notably to stimulate antigen presentation. Pis may affect antigen processing for presentation within class I MHC, production of inflammatory cytokines or NF-kB activation (Kourjian et al., (2014). J Immunol. Apr 15;192(8):3496-506).

### Cytokine Release Syndrome

Cytokines participate in many physiological processes including the regulation of immune and inflammatory responses. These effector molecules are produced transiently and locally controlling the amplitude and duration of the response. A variety of experiments has shown that excessive or insufficient production may significantly contribute to the pathophysiology of a range of diseases. Quantification of cytokines has significant value in both clinical medicine and biology as the levels provide insights into physiological and pathological processes and can be used to aid diagnosis and treatment. Cytokines and their clinical significance are introduced from the perspective of their pro-and anti-inflammatory effects.

Disclosed herein are methods of treatment or prevention of diseases with cytokine release syndrome (CRS, also referred to as “cytokine storm”) by administration with the protease inhibitor provided herein. CRS is an acute systemic inflammatory syndrome from immune cell-based therapies, e.g., CAR T cell treatment, therapeutic antibodies, and haploidentical allogeneic transplantation (such as organ transplantation). Severe CRS is a potentially life-threatening toxicity. Toxicities primarily manifest as cytokine release syndrome characterized by high elevations of cytokines including INFγ, granulocyte-macrophage colony-stimulating factor, IL-10, and IL-6. These cytokine elevations result in a plethora of clinical symptoms including fever, hypotension, organ dysfunction, respiratory failure and coagulopathy. CRS can be fatal. Additionally, neurotoxicity often presents even after the initial symptoms of CRS have subsided. The pathogenesis of CRS and associated neurotoxicity is poorly understood and further understanding of the mechanism would be useful for the successful translation of CAR-T therapy. In the meanwhile, disrupting the pathogenesis of CRS by reducing the level of cytokine is one way to mitigate the condition.

In certain embodiments, the subject has cytokine release syndrome. In certain embodiments, the subject is a patient has received or is prescribed for organ transplantation. In certain embodiments, the subject has received or is prescribed for an immune cell therapy, such as an allogeneic or autologous T cell, NK cell, or macrophage therapy. The cell may express a chimeric antigen receptor or a recombinant T cell receptor.

### Skin Disorders

The skin is the largest organ in the human body, representing 16% of the total body weight. The skin consists of an outer layer (termed the epidermis) that enables the body to interact with the environment through physico-chemical mechanisms and sophisticated sensorial stimuli. Moreover, the epidermis provides protection for the human body through specialized cells involved in immunity, which are distributed throughout the organ.

The skin inflammation includes acute skin inflammation and chronic skin inflammation. The acute skin inflammation includes sunburn, damage due to heat exposure, acne, blisters, hives (urticaria), allergic reaction (contact dermatitis), insect bite reaction, and bacterial, viral, or fungal infection. The chronic inflammation includes eczema (e.g., atopic, dyshidrotic, or seborrheic dermatitis), psoriasis (e.g., plaque, pustular, or inverse psoriasis), rosacea (e.g., phymatous or papulopustular rosacea), dermatomyositis, impetigo (non-bullous and bullous type), and vitiligo (generalized and non-segmental type), hand dermatitis, lichen planus, drug hypersensitivity reactions, lichen simplex chronicus, prurigo nodularis, cutaneous T cell lymphoma, blistering autoimmune diseases (pemphigus family of disorders or bullous pemphigoid/pemphigoid family of disorders), or retinoid dermatitis.

In certain embodiments, the skin disorder includes skin inflammation. In certain embodiments, the skin inflammation includes psoriasis.

Psoriasis is a chronic inflammatory skin disease characterized by skin thickening and sharply demarcated erythematous plaques covered by silvery white scales due to hyperproliferation and excessive accumulation of epidermal keratinocytes. Psoriasis encompasses dysregulation and complex interactions of the innate and the adaptive immune systems as well as other components such as the epithelium, the vasculature and the cutaneous nervous system. There is accumulating evidence that both autoimmune and autoinflammatory mechanisms fuel the disease. Although the precise mechanism is still not fully elucidated, it is commonly accepted that dysregulated auto-reactive immune system is involved in the pathogenesis of this chronic skin disease.

Common therapeutic agents to treat the skin inflammation include anti-histamines, non-steroidal anti-inflammatory drugs (NSAIDs), antibiotics, antifungal medication, immunosuppressants, moisturizers, creams, and serums of antioxidant agents, corticosteroids, immunomodulators, light therapy, such as red light therapy, blue light therapy. Such therapeutic agents can be used in a second therapy in addition to the protease inhibitor treatment provided herein.

### Cancers

In certain embodiments, the subject has cancer. In some embodiments, provided are methods for treating a cancer in a patient in need thereof. The method, in one embodiment, entails administering to the patient an effective amount of a protease inhibitor of the present disclosure.

In some embodiments, provided are uses of the protease inhibitors of the present disclosure in the manufacture of a medicament for treating a cancer in a patient in need thereof.

In some embodiments, provided are the protease inhibitor of the present disclosure for use in the treatment of a cancer in a patient in need thereof.

The cancer can be a solid tumor or hematologic malignancies. Non-limiting examples of cancers include bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, esophageal cancer, ovarian cancer, renal cancer, melanoma, skin cancer, prostate cancer and thyroid cancer.

### EXAMPLES

### Example 1. Effects of Paxlovid in Immune Cells

Observation with Paxlovid: Surprisingly, a patient was observed with complete clearance and cessation of symptoms of a chronic and persistent hand dermatitis under treatment of COVID-19 with Paxlovid, a drug developed to block the SARS-CoV-2 replication. Therefore, Paxlovid might alter immune cell function to impact a host of diseases include skin inflammation and skin cancer, as well as inflammation and cancers for other organ systems.

### Materials and Methods

### Healthy Blood Donors

For this study, peripheral blood for all experiments was acquired from healthy human donors enrolled in The Lundquist Research Institute IRB-approved protocol (Collection of Peripheral Blood from Healthy Volunteers to Investigate Mechanisms of Innate and Adaptive Immunity IRB #: 31121-01). In all cases, written informed consent was obtained. All subjects gave written informed consent in accordance with the Declaration of Helsinki.

### Preparation and Culture of Peripheral Blood Mononuclear Cells and Monocytes

For all experiments, peripheral blood mononuclear cells (PBMC) were isolated either from healthy blood donors as above or purchased from HemaCare as Leukopaks (D186, D317 and D93). PBMC were purified by Ficoll-Hypaque gradient centrifugation (Pharmacia, Piscataway, NJ) and stimulated after freshly isolated as described, or cryopreserved in cryoprotective medium consisting of 90% fetal bovine serum (FBS) in combination with 10% dimethyl sulfoxide (DMSO) at 20 to 30 million cells per vial, and thawed for later stimulation as described. For studies with enriched monocytes, PBMC were cultured in 1% FBS in RPMI 1640, and adhered to sterile plastic tissue culture plates for 1-2 hours; followed by washing off nonadherent PBMC. Remaining adherent monocytes were stimulated as described.

### Bacterial Preparations Used for Stimulation

Bacteria were grown in appropriate medium: Nutrient medium (Methylobacterium radiotolerans), Tryptic Soy Medium (Sphingomonas yanoikuyae), and reinforced Clostridial Medium (Cutibacterium acnes, Cutibacterium granulosum). Cells were harvested in log phase and sonicated in PBS at 10 ng/ml. After sonication of the bacteria, bacterial protein concentration was measured using the Bio-Rad Protein Assay (Hercules, California) to normalize the amount of bacteria added.

### Stimulation of Immune Cells

Lipopolysaccharide (LPS) is a key component of gram-negative bacteria outer walls and a contributing factor to the toxicity of many bacterial species (Wang et al., Prog Lipid Res. 2010 Apr;49(2):97-107). LPS itself can stimulate a strong inflammatory response leading to transcription of inflammatory cytokines. It is considered a common laboratory reagent for inflammation studies in culture medium or injected into animal models to stimulate bacterial infection (Lu et al., Cytokine. 2008 May;42(2):145-151; Park et al., Mol Med. 2013 Dec 6;45(12):e66).

PBMC or monocytes were stimulated with LPS (10 ng/ml) or bacterial sonicates at 0.1 g/ml concentration +/−added drugs. To study effects on activated T cells, PBMC were stimulated with anti-CD3/CD28 (beads or plate-bound anti-CD3 and soluble anti-CD28)+/−addition of drugs. Conditioned media were harvested after overnight culture (16-24 hours) and assayed for various cytokines (IL-1β, IL-2, IL-4, IL-6, IL-10, IL-23, IFNγ, TNF-α) by ELISA (BD PharMingen, San Diego, CA) per manufacturer's instructions and ELISA for IL-23 was done per manufacturer's instructions by R&D Systems Minneapolis, MN. All Conditioned media samples were assayed by ELISA in duplicate. Standard curves and individual well concentrations were determined using the Softmax 2.34 software (Molecular Devices, Menlo Park, CA). To keep experimental values within the linear region of the standard curves, samples were diluted as necessary. Conditioned media were diluted to 1:3, 1:9 and 1:27 to have (IL)-1β within standard curve. Conditioned media were diluted to 1:6, 1:36 and 1:216 for IL-6 to fit within standard curve. Conditioned media were tested neat and at 1:2 dilutions for IL-10. Conditioned media were not diluted to detect IL-23. Conditioned media were tested neat, and at 1:3 and 1:9 dilutions to detect tumor necrosis factor TNF-α.

After activating T cells, the conditioned media were diluted to 1:3, 1:9 and 1:27 to determine IFNγ concentration, and IL-2 concentration. Undiluted conditioned media were tested to determine IL-4 concentration. Conditioned media were tested neat and at 1:2 dilutions to determine IL-10 concentration. Conditioned media were tested undiluted, or diluted 1:3 and 1:9 to determine tumor necrosis factor (TNF) α concentration.

### Monocyte Enrichment

Isolated PBMCs were washed once in 1% FCS RPMI and re-suspended in RPMI-1640 (ThermoFisher Scientific) in 1-2% FCS RPMI. For monocyte isolation, 2×10⁶ PBMCs per well were plated in 24-well tissue culture treated plate (ThermoFisher Scientific) at 1 ml each well. Monocytes were allowed to adhere in a 5% CO2 container at 37° C. for 2 hr. Nonadherent cells were removed by thorough washing 2 times with 1% FCS RPMI-1640.

### Concentrations of RTV, NMV and NMV+RTV

NMV, RTV and NMV+RTV were dissolved in water-free DMSO at 5 mg/ml stock concentration and stored in single-use aliquots at −80° C. We tested three different concentrations 1250 ng/mL, 2500 ng/ml and 5000 ng/ml to evaluate the toxicity on cells.

### Flow Cytometry

To test the effect on death/apoptosis of the PBMCs we tested three concentrations of NMV and RIT on peripheral blood mononuclear cells (PBMC) from three different donors (D186, D317 and D93). Labeling of PBMCs was performed using CD3 (FITC-Invitrogen), CD14 (APC-BD pharmigen) according to the manufacturer's instructions. Cell viability was assessed by staining with Propidium Iodide Cell Viability-PI (BD-Pharmigen). Flow cytometry was performed using a BD FACSymphony A5 and analyzed with FlowJo 10.1 software (FlowJo LLC, OR, USA).

### CCK-8 Viability Test

Cell Counting Kit-8 (CCK-8) is a premixed, ready-to-use colorimetric assay used for determining the number of viable cells in a culture well. For these experiments cells were plated in a 96 well-plate at density of 25,000 cells per well in 100 μL of culture medium according to manufacturer's instructions (Dojindo laboratories—Rockville, MD). Two million cells were incubated in 5% CO2 at 37° C. for 24 hours. 10 μL of CCK-8 solution was added to each well and incubated for 3 hours. Optical density at 450 nm was measured using the Synergy H1 Hybrid Multi-Mode Reader.

### RNA Sequencing and Statistics

PBMC from four healthy donors were cultured in 48 well plates, two million cells per well. For effects of NMV+RTV on myeloid stimulated cells, the PBMC were cultured in 10% FBS in RPMI 1640 in the presence of LPS at 10 ng/ml+/−NMV+RTV two different doses (2500 ng/ml for each drug or 5000 ng/ml for each drug) for a total of four conditions. For effects of NMV+RTV on activated T cells, the PBMC were cultured in 10% FBS in RPMI 1640 in the presence of 2 μL αCD 3/CD28 beads+/−NMV+RTV two different doses (2500 ng/ml for each drug or 5000 ng/ml for each drug) for a total of four conditions.

Library preparation was performed by Novogene using NEBNext Ultra II RNA Library Prep by Illumina. Samples were sequenced on NovaSeq 6000. Q-scores<=5 and reads containing >10% N bases (ambiguous) were removed. Raw reads were then trimmed using default options with Trimmomatic v0.36. Reads were mapped against the GRCh38.p12.genome using hisat2 aligner and counted using htseq. Gene IDs with average counts per million (CPM) less than five across the samples were excluded before TMM normalization of raw counts. Normalized counts were then used as input for differential gene expression analysis using limma-voom, which additionally were adjusted for correlation between the same subjects before applying empirical Bayes for a moderated t-test. Genes were considered statistically significant under P<0.05, and their common Ensembl gene identifiers were used as input for over-representation analysis (ORA) using Fisher's exact test against the KEGG database on WebGestalt.

Ordination plots were performed on TMM normalized count data using t-SNE algorithm with perplexity of six (seed 888). PERMANOVA test was used to compare whether samples clustered significantly by treatment conditions.

Cytokine dose-response analysis was performed using linear regression model testing dose (ordinal variable) and adjusting for the individual donors.

### Results

### Ritonavir and Nirmatrelvir Do Not Affect Peripheral Blood Mononuclear Cells' Viability

To test the viability of cells we performed flow cytometer and CCK-8 assays using three different concentrations of NMV and RTV: 1250 ng/ml, 2250 ng/ml and 5000 ng/ml.

In particular, gating strategy for flow cytometry analysis to evaluate viable T cells and monocytes using concentrations of NMV (1250, 2500 and 5000 ng) for treatment during 24 hrs in frozen blood from three different donors. Debris were excluded, analysis was done using a forward scatter area (FSC-A) versus side scatter area (SSC-A) gate. T cells (CD3+) population labeled with Fluorochrome FITC versus PI live/dead fluorochrome were shown on histogram analysis (1B) treated with three different concentrations of NMV. Monocytes population (CD14+) labeled with fluorochrome APC versus Propidium iodide (PI) fluorochrome were shown on histogram analysis (1C) treated with three different concentrations of NMV.

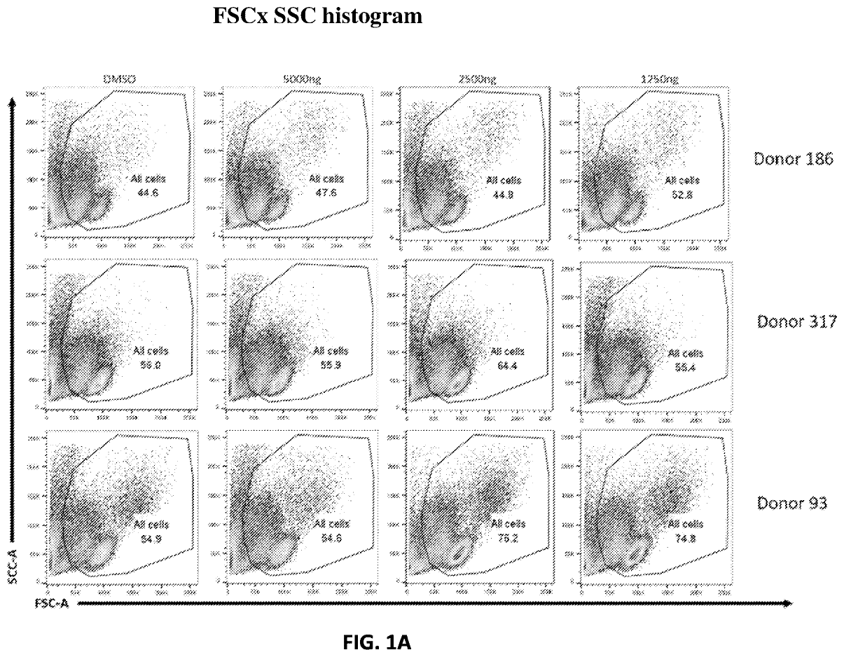

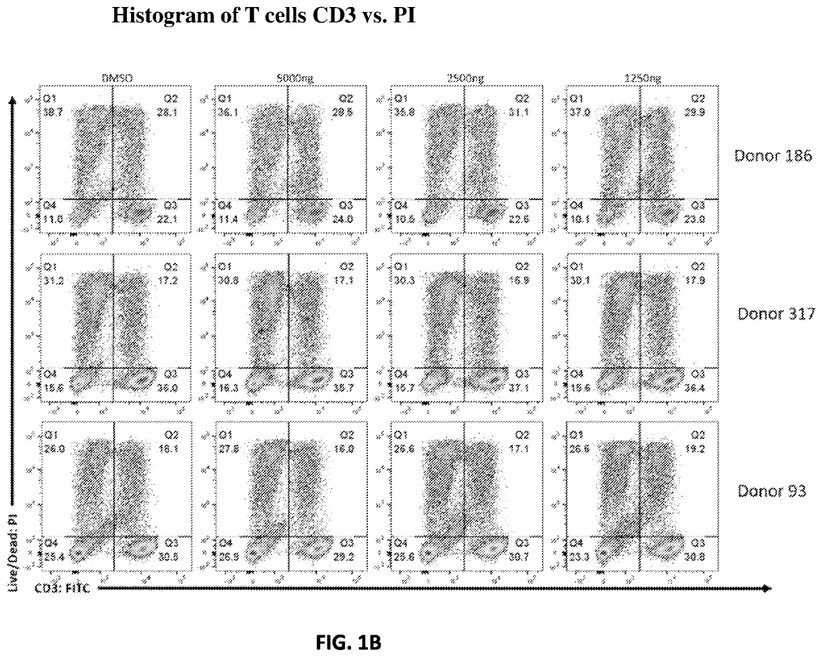

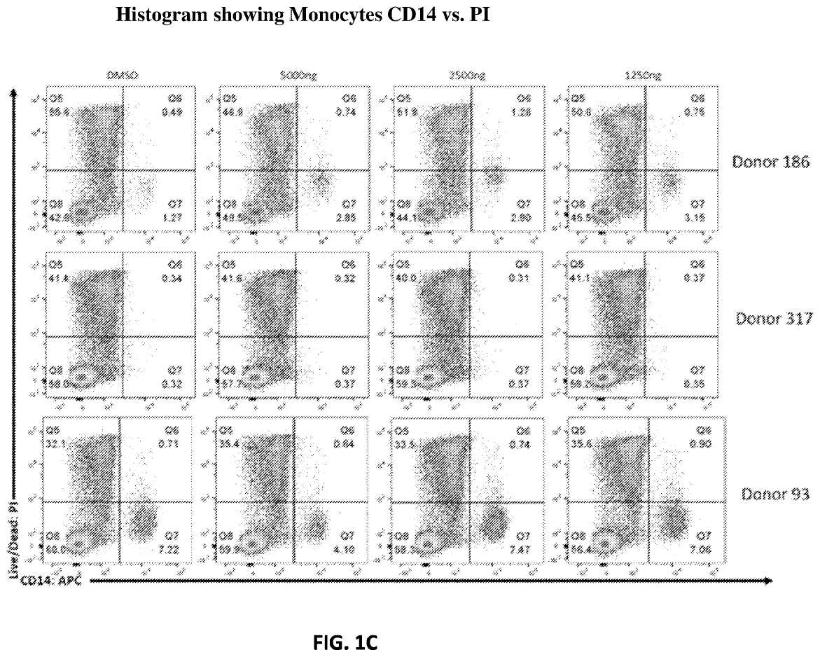

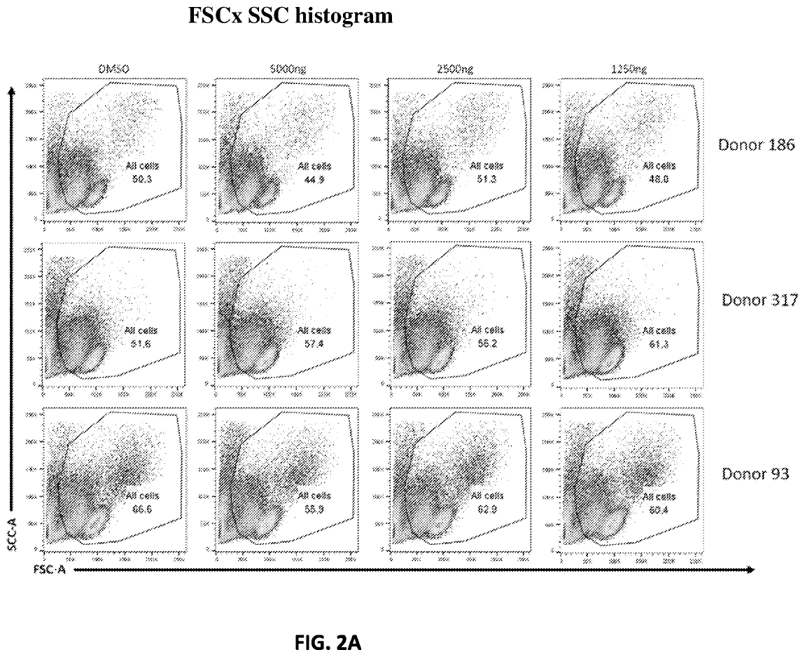

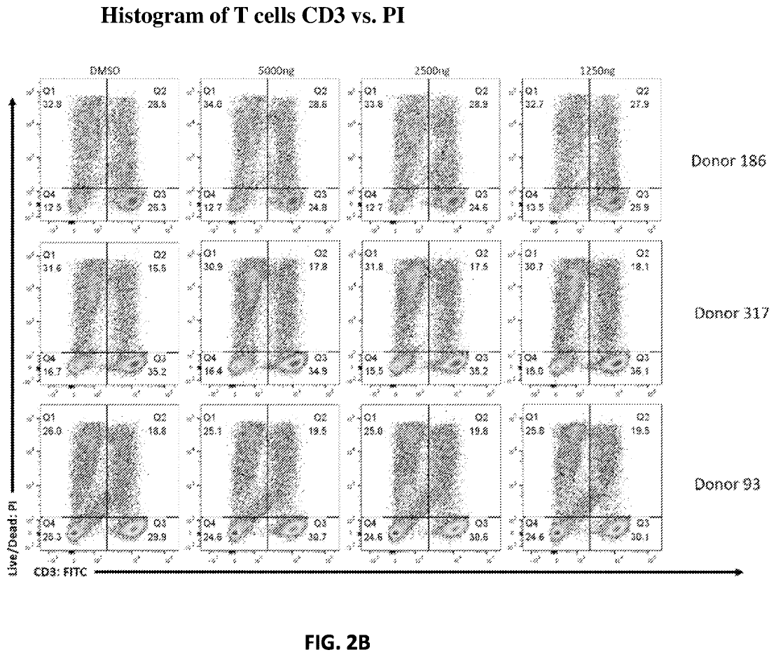

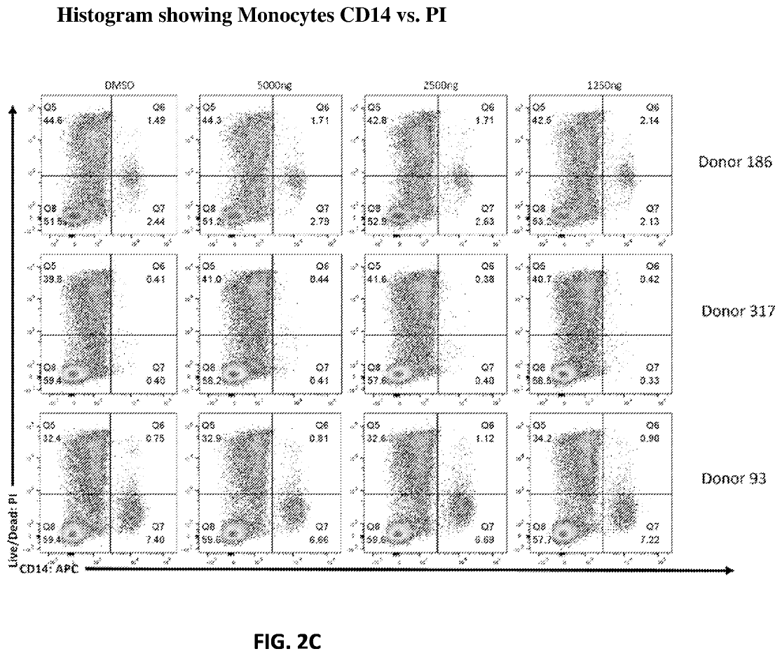

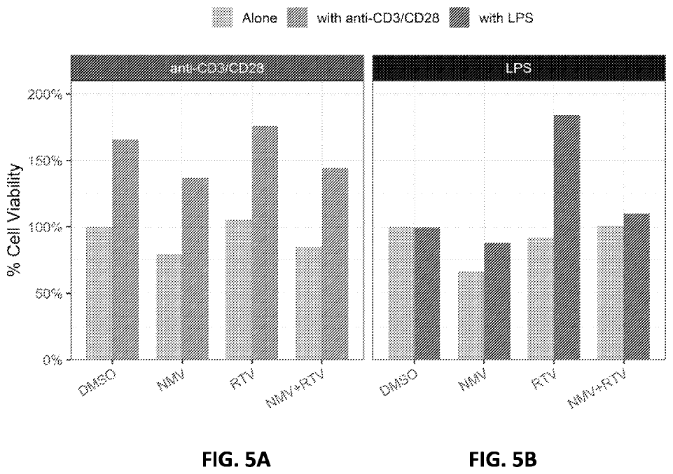

We found that all different concentrations did not affect the viability of PBMCs. To test the effect of NMV, we selected PBMCs (FIG. 1A) and gated the CD3+ cells to select T cells (FIG. 1B) and CD14+ cells to select monocytes (FIG. 1C) through Flow Cytometer. The detection showed similar toxicity in the cells with concentrations of NMV when compared to DMSO treatment only. The same methodology using PBMCs from the same donors (D186, D317, and D93) was performed to assess the toxicity of RTV (FIG. 2A). With 1250, 2000, and 5000 ng/ml of RTV, the gated CD3+ T cells (FIG. 2B) and the gated CD14+ Monocytes (FIG. 2C) showed similar toxicity for all three donors. To validate the results, we decided to use Frozen Leukopak PBMCs (which are cells collected via leukapheresis and can supply studies with up to 20 billion PBMCs per leukopak). CCK-8 (Cell Counting Kit-8) viability assay was performed for anti-CD3/CD28 beads activated T cells (FIG. 5A) and for LPS stimulated PBMCs (FIG. 5B) treated with NMV, RTV, and NMV+RTV at 5000 ng/mL.

### Nirmatrelvir Decreased Inflammatory Cytokines Except IL-1β

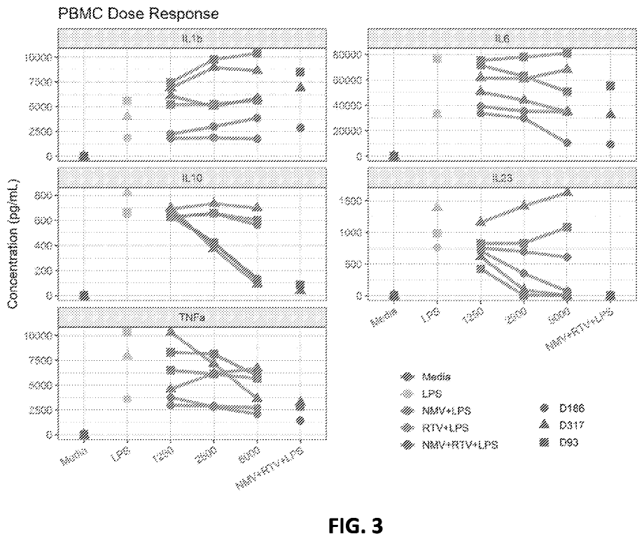

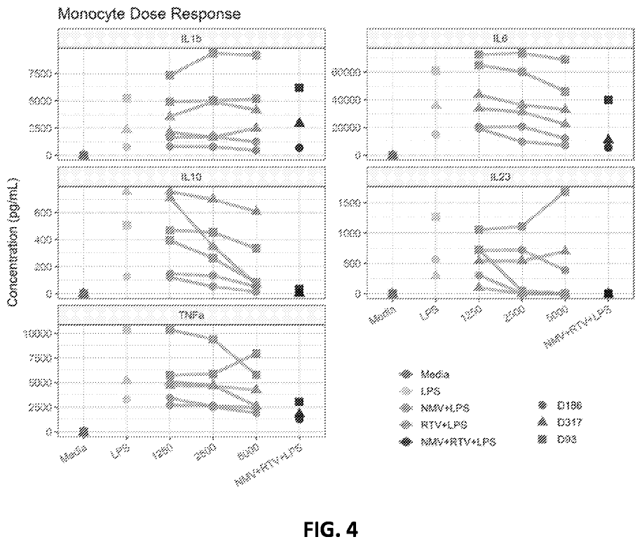

To investigate the dose response of NMV and RTV on pro and anti-inflammatory cytokines in frozen PBMCs stimulated with LPS (same donors as Flow Cytometer), ELISA assay was performed to evaluate levels of IL-1β, IL-6, IL-10, IL-23 and TNF-α when treated with NMV and RTV separately (at 1250, 2250, and 5000 ng/ml), and when treated with NMV+RTV together (5000 ng/ml) (FIG. 3). NMV was able to decrease cytokines except IL-1β. Same results were observed when we treated the cells with NMV+RTV. To attest our results with PBMCs, we performed ELISA assay in monocytes (FIG. 4) and the results were consistent, showing decreasing levels of IL-6, IL-23, IL-10, TNF-α, while IL-1β was decreased when monocytes were treated with 5000 ng/ml of RTV. PBMC from donor D186 showed low levels of IL-1β.

Since the results were consistent among the different donors, we decided to use the concentration 5000 ng/mL of NMV+RTV together, as it did not show toxicity in the cells stimulated with LPS compared to DMSO (FIG. 5). Concentrations used for NMV were able to decrease levels of pro and anti-inflammatory cytokines. IL-1β, a pro-inflammatory cytokine was elevated when treated with RTV and when treated with NMV kept the same levels when compared to LPS stimulation only group. Such results can be variable since different donors might present different healthy conditions when the blood was collected.

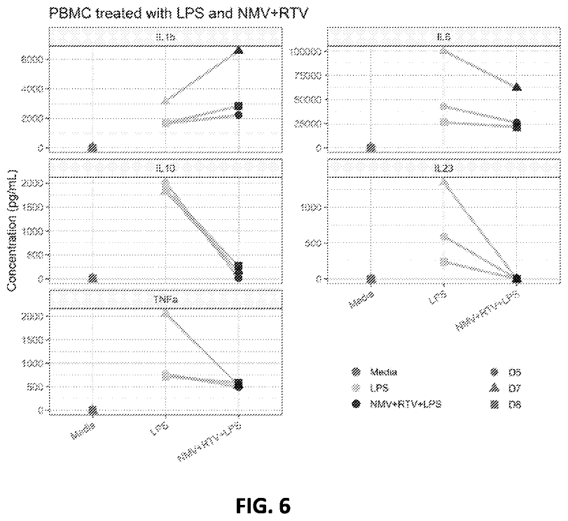

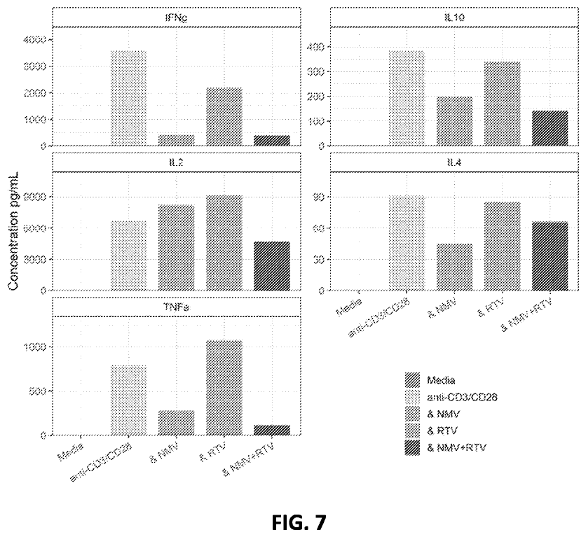

Having validated the findings using Leukopak PBMCs and frozen blood from three independent donors, another test-set of three different donors were used to assess cytokine levels in LPS stimulated cells treated with NMV+RTV at 5000 ng/ml (FIG. 6). ELISA was performed, showing low levels of IL-6, IL-10, IL-23, TNF-α, and higher levels of IL-13 when treated with 5000 ng/ml of NMV+RTV in cells stimulated with LPS, corroborating the results above. Donor 007 was used to test levels of IL-2, IL-4, IFN-γ, IL-10 and TNF-α cytokines in T cells activated using beads in a manner that partially mimics stimulation by antigen-presenting cells (FIG. 7). Data showed low levels of IFN-γ, a pro-inflammatory cytokine secreted by T cells that plays a role in regulating the immune response. TNF-α was also low when cells were treated with NMV and N+R.

### Nirmatrelvir and Ritonavir Affect Cytokine Secretion by Human PBMC when Stimulated with Gram-Positive and Gram-Negative Bacteria

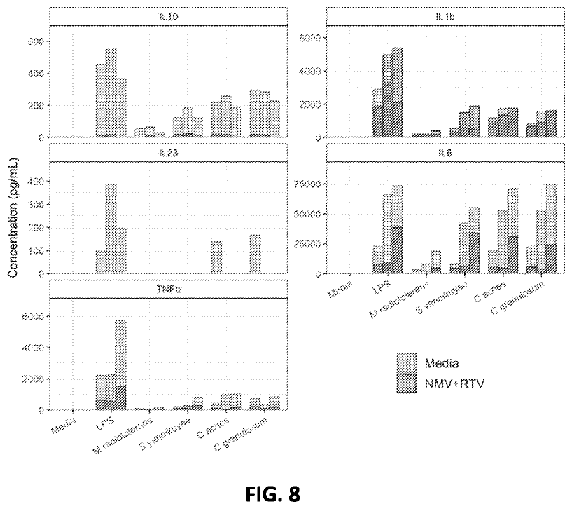

PBMCs produce a wide array of cytokines in response to microbial pattern recognition ligands. When we added sonicated bacteria (M. radiotolerans, S. yanoikuyae, C. acnes and C. granulosum) to the cells and treated with NMV+RTV at 5000 ng/ml (FIG. 8), we observed decreased secretion of IL-6, TNF-α, IL-10 and IL-23 inflammatory cytokines when treated with NMV and RTV. IL-1β showed higher levels again when compared to the other cytokines evaluated in this study.

### RNA Sequencing

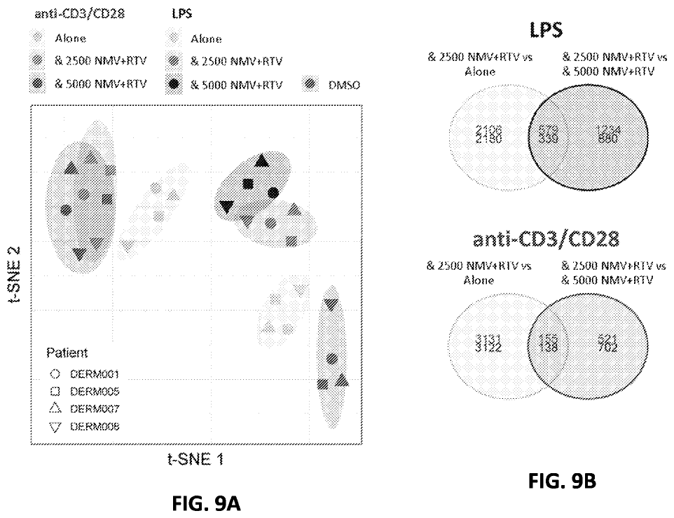

To further investigate the transcriptomic effects of NMV+RTV on LPS-activated PBMC and on ant-CD3/28 T cell activated PBMC, we performed bulk RNA sequencing on PBMC from four independent subjects. Cells were stimulated in vitro in the same manner as the above studies in which we measured cytokines with LPS or anti-CD3/CD28 beads in the presence or absence of NMV+RTV (2500 ng/ml or 5000 ng/ml of each drug). Analysis using t-SNE ordination show that the gene expression profiles mostly cluster by treatment, and importantly they did not cluster by individual subjects (FIG. 9A). Furthermore, there is strong clustering between LPS alone and anti-CD3/CD28 alone versus when in conjunction with Pax at 2500 ng/ml (PERMANOVA P<0.001 for both). Clustering is not clear when increasing the dose of NMV+RTV from 2500 to 5000 ng/ml with anti-CD3/CD28 (overlaps in purple shades; the two groups show no significant differences in their centroids and dispersion based on Euclidean distance matrix; PERMANOVA P=0.283) and was noisier (larger dispersion) when with LPS (the blue points spread along x-axis [t-SNE1], P=0.084).

Pair-wise differential analyses were performed to identify genes that increased or decreased in a dose-responsive manner with increasing or decreasing expression with higher doses of NMV+RTV. Genes that are differentially increased or decreased in expression by NMV+RTV (2500 ng/ml of each drug) are further increased or decreased by the higher dose of NMV+RTV (5000 ng/ml of each drug) (FIG. 9B). The resulting overlapping gene lists from the two experiments (one with LPS and one with anti-CD3/CD28) are then used for pathway over-representation analysis; assessing differentially increased or decreased transcribed genes involved in pathways associated with NMV+RTV treatment to identify potential effects on biological processes or pathways. We found KEGG pathways over-represented with gene sets statistically increased or decreased by NMV+RTV on LPS-stimulated cells and on anti-CD3/CD28 stimulated cells.

### Example 2. Effects of Paxlovid in Treating Allergy and Psoriasis

This example reports further findings relating to the therapeutic effects of paxlovid in treating allergy and psoriasis, an autoimmune disease.

In the clinic, a patient with with severe allergy and Covid 19 was treated with paxlovid. Following the treatment, the severe allergy was completely resolved. Another patient, who had mild psoriasis and Covid 19, was also treated with paxlovid. Following the treatment, the psoriasis symptoms were alleviated. For both patients, the symptoms were alleviated for months even though the paxlovid treatment was for only 5 days.

### Example 3. Effects of Paxlovid for Novel Skin Disease Therapy

Given these effects of NMV and RTV in vitro, NMV+RTV may be helpful in the treatment or management of inflammatory immune mediated skin diseases. For this, a common animal model for the study of immune-related skin diseases is the imiquimod (IMQ)-induced psoriasis animal model. The IMQ-induced inflammatory skin mouse model has features of skin inflammatory diseases similar to skin inflammation observed in human psoriasis.

Many insights into psoriasis-in particular into the efficacy of therapies and into complex interactions of immune cells and inflammatory mediators-come from animal models, especially in mice. Topical application of imiquimod (IMQ), a toll-like receptor (TLR)-7 and TLR-8 agonist, induces psoriasis-like skin inflammation in mice. Furthermore, the Dendritic cell (DC)/Th1/TH17 axis plays an important role in developing skin inflammation. The antiviral and antitumor effects of IMQ are mostly mediated via activation of TLR7 and TLR8 expressed by monocytes, macrophages, and pDC. The production of proinflammatory cytokines and chemokines is increased, directly resulting in antiviral activity mediated by IFN-α and in influx of immune cells to the site of application.

To test the hypothesis that NMV+RTV might improve to psoriasis, the effect of NMV+RTV on imiquimod (IMQ)-induced skin inflammation in mice will be tested. In this study, to confirm and further extend the pharmacological basis of NMV and RTV in psoriasis therapy, the effect of NMV+RTV on psoriasis-like skin inflammation in mice induced by IMQ will be investigated. The effects of pharmacological activity of NMV and RTV in mouse models of inflammatory skin conditions will be investigated. Unpublished in vitro data suggests that NMV+RTV may dampen proinflammatory cytokine production, the effect in an in vivo model of skin inflammation will be tested.

In vivo models of inflammatory skin disorders used for preclinical studies of compound efficacy have unique strengths and limitations. The use of animal models for drug discovery has increased at an exponential rate in recent years. The body surface area of skin in adults covers an area of 1.5-2.0 m² and accounts for almost 15% of the body weight. It provides resistance against trauma, provides protection against infections, prevents body from losing excess water, helps regulate the body temperature, synthesizes Vitamin D, and permits sensations of touch, heat, and cold. Skin constitutes the largest biological barrier that protects from dehydration and mechanical injury, environmental toxins and microbes, and participates in immune responses. The epidermis of the skin is comprised of multilayered and stratified epithelial cells that undergo life-long self-renewal through a tightly regulated balance of cell proliferation and terminal differentiation. Disruption of this balance represents a central process of a myriad of skin diseases.

Animal models promote a better understanding of skin disorders, they are of great utility to investigate inflammatory mediators, the communication between cells of the innate and the adaptive immune systems, the role of resident cells as well as the use of new drugs and therapies.

Because basic and translational research on patients with psoriasis is often not possible due to ethical considerations and the very limited availability of human material, animal models are of great importance for the investigation of pathogenic mechanisms and therapeutic principles.

NMV and RTV will decrease skin inflammation in vivo in the Psoriasis animal model Imiquimod (IMQ)-induced skin inflammation in mice.

The objective is to utilize findings from these exploratory studies to continue the academic discovery and help in the translational efforts towards finding a way to cure or restrain the damage caused by poor immune function to cause inflammation in autoimmune and inflammatory diseases. While manipulating skin inflammation is a clear first step, the applications of this drug whether administered systemically (by oral routes, intravenously, subcutaneously) or locally (by intralesional injection or topical formulations) has potential for being farther reaching than just in skin disease. As mentioned above, sepsis (and septic shock) which are life threatening conditions, caused by gram-negative bacteria via the release of the cell-wall component known as endotoxin (lipopolysaccharide-LPS) is an obvious condition for which NMV+RTV or related compounds may impact given the findings we have on modulation of inflammatory cytokines induced by LPS. In fact, we also are planning experiments utilizing libraries which cover FDA approved drugs for re-purposing, chemical genomics libraries for dissecting biological pathways, various larger libraries for drug discovery, as well as libraries of drugs in clinical trials such as kinase inhibitors. Therefore, the findings that NMV+RTV or related compounds may regulate inflammation has far reaching potential and implications on improving inflammatory diseases and autoimmunity.

### Example 4. Selective Killing of Tumor Cells

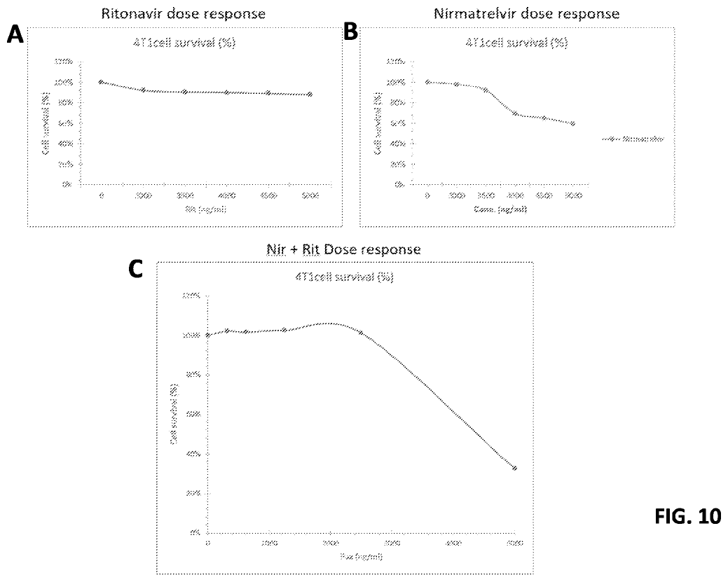

This example shows that Ritonavir+Nirmatrelvir or Nirmatrelvir alone killed 4T1 cells in a dose responsive manner (FIG. 10). 4T1 is a breast cancer cell line derived from the mammary gland tissue of a mouse BALB/c strain. 4T1 cells are epithelial and are resistant to 6-thioguanine. In preclinical research, 4T1 cells have been used to study breast cancer metastasis as they can metastasize to the lung, liver, lymph nodes, brain and bone.

The treatment of a mouse model of breast cancer using 4T1 cells will show delayed tumor progression and/or regression of tumor and/or cure after administration of Ritonavir+Nirmatrelvir or Nirmatrelvir alone in different modes of delivery (oral, intraperitoneal).

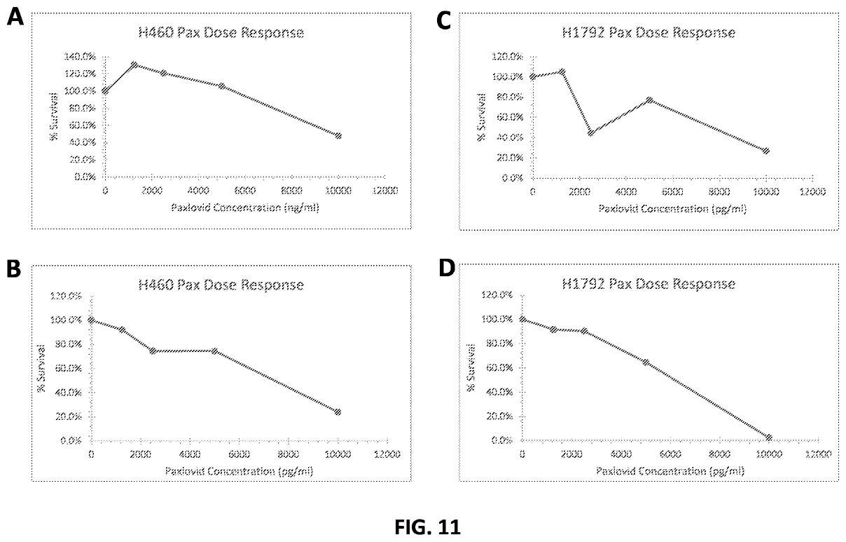

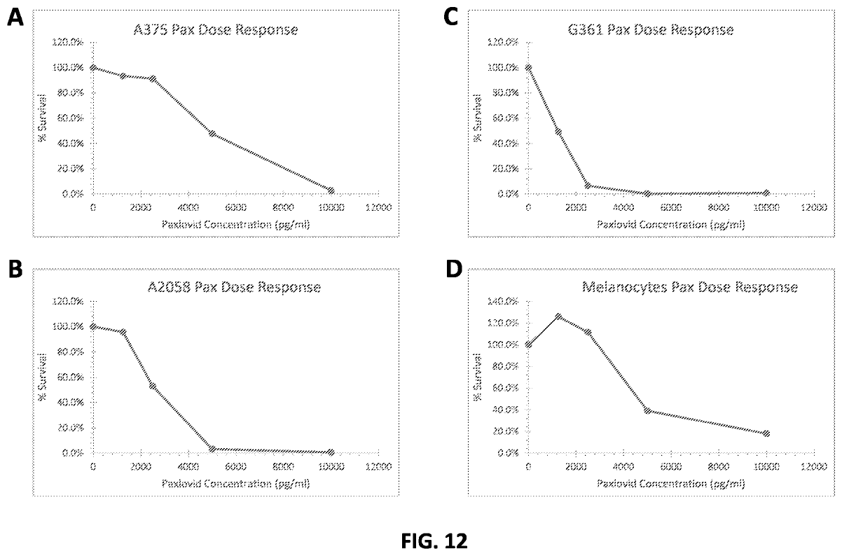

The dosage response manner was also observed in lung cancer cell lines H460 and H1792 (FIG. 11) and melanoma cell lines A375, G361 and A2058 (FIG. 12).

Gene expression profile of the cancer cell lines (sensitive versus resistant) were performed. The results demonstrate excellent therapeutic index for cancer treatments.

### * * *

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for reducing immunity, suppressing or modulating immune reaction, or preventing or treating cancer, an inflammatory disorder, or an autoimmune or autoinflammatory disease or condition in a subject in need thereof, comprising administering to the subject a protease inhibitor.

2. The method of claim 1, wherein the protease inhibitor is a viral protease inhibitor.

3. The method of claim 1, wherein the protease inhibitor is a 3-chymotrypsin-like protease inhibitor.

4. The method of claim 1, wherein the protease inhibitor is nirmatrelvir, ensitrelvir, boceprevir, paritaprevir, ombitasvir, tipranavir, ivermectin, simnotrelvir, and micafungin.

5. The method of claim 1, further comprising administering to the subject a CYP3A4 inhibitor.

6. The method of claim 5, wherein the CYP3A4 inhibitor is selected from the group consisting of amiodarone, amlodipine, amprenavir, aprepitant, atazanavir, azamulin, berberine, berberis, bergamottin, bicalutamide, boceprevir, buprenorphine, cafestol, cannabidiol, carambola, ceritinib, chloramphenicol, chlorzoxazone, ciclosporin, cilostazol, cimetidine, ciprofloxacin, clarithromycin, clotrimazole, cobicistat, conivaptan, crizotinib, delavirdine, 6′,7′-dihydroxybergamottin, diltiazem, dithiocarbamate, dronedarone, econazole, erythromycin, fluconazole, fluvoxamine, fosamprenavir, fosaprepitant, gestodene, idelalisib, imatinib, indinavir, isavuconazonium, isoniazid, istradefylline, itraconazole, ivacaftor, ketoconazole, levoketoconazole, lomitapide, lopinavir, methoxsalen, mibefradil, miconazole, mifepristone, nefazodone, nelfinavir, niacin, nicotinamide, norfloxacin, omeprazole, orphenadrine, piperine, posaconazole, quercetin, ranitidine, ranolazine, ribociclib, ritonavir, rutin, saquinavir, sesamin, silybum marianum, tacrolimus, telaprevir, telithromycin, ticagrelor, tofisopam, troleandomycin, tucatinib, valerian, valproate, verapamil, vitamin B3, and voriconazole.

7. The method of claim 6, wherein the CYP3A4 inhibitor is ritonavir.

8. The method of claim 7, wherein the method comprises administering to the subject nirmatrelvir and ritonavir.

9. The method of claim 1, wherein the subject is an organ transplantation patient.

10. The method of claim 1, wherein the subject has received or is prescribed for an immune cell therapy, preferably selected from an allogeneic or autologous T cell, NK cell, or macrophage therapy.

11. The method of claim 10, wherein the cell comprises a chimeric antigen receptor or a recombinant T cell receptor.

12. The method of claim 1, wherein the subject has cytokine release syndrome.

13. The method of claim 1, wherein the subject has an autoimmune or autoinflammatory disease.

14. The method of claim 13, wherein the autoimmune disease is selected from the group consisting of rheumatoid arthritis, systemic lupus erythematosus (SLE), multiple sclerosis (MS), atherosclerosis, amyotrophic lateral sclerosis, type 1 diabetes, psoriasis, atopic dermatitis, contact dermatitis and eczematous dermatitis, osteoarthritis, allergic airway disease (e.g. asthma), chronic obstructive pulmonary disease (COPD), inflammatory bowel disease (IBD), Crohn's disease, ulcerative colitis, cutaneous lupus erythrematosis, lupus nephritis, dermatomyositis, dry eye disease, Sjögren's syndrome, Myasthenia gravis, Grave's disease, Hashimoto's thyroiditis, Celiac disease, Pernicious anemia, autoimmune hemolytic anemia, Goodpasture syndrome, dermatomyositis, polymyositis, and Addison's disease.

15. The method of claim 1, wherein the subject has acute or chronic inflammation.

16. The method of claim 15, wherein the acute or chronic inflammation is in the skin.

17. The method of claim 16, wherein the acute or chronic inflammation is (i) an infectious disease, preferably selected from the group consisting of bacterial infection, viral infection and fungal infection, (ii) eczema, preferably selected from the group consisting of atopic, dyshidrotic, and seborrheic dermatitis), (iii) psoriasis, preferably selected from the group consisting of plaque, pustular, and inverse psoriasis, (iv) rosacea, preferably selected from the group consisting of phymatous and papulopustular rosacea, (v) dermatomyositis, (vi) impetigo, (vii) vitiligo, (viii) hand dermatitis, (ix) lichen planus, (x) drug hypersensitivity reactions, (xi) lichen simplex chronicus, (xii) prurigo nodularis, (xiii) cutaneous T cell lymphoma, (xiv) blistering autoimmune disease, preferably selected from the group consisting of pemphigus family of disorders and bullous pemphigoid/pemphigoid family of disorders, or (xv) retinoid dermatitis.

18. The method of claim 16, further comprising topical administration of an agent selected from the group consisting of a moisturizer, a corticosteroid, an immunomodulator, dapsone, inhibitors of the JAK-STAT pathway, inhibitors of the IL-17 pathway, inhibitors of the cytokine-cytokine receptor interaction, cyclooxygenase inhibitors, a macrolide antibiotic preferably selected from tacrolimus or pimecrolimus, inhibitors of PDE preferably selected from crisoborale or apremilast, a retinoid preferably selected from tretinoin, retinol, retinaldehyde, tazarotene or adapalene, and combinations thereof.

19. (canceled)

20. (canceled)

21. The method of claim 1, wherein the subject has cancer.

22. (canceled)

23. (canceled)

24. The method of claim 21, wherein the cancer is selected from the group consisting of bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, esophageal cancer, ovarian cancer, renal cancer, melanoma, skin cancer, prostate cancer and thyroid cancer.
